(19) United States
(12) Patent Application Publication
(10) Pub. No.: US 2025/0375551 A1
(43) Pub. Date: Dec. 11, 2025

(54) ULTRAVIOLET (UV) LAMP ASSEMBLY

(71) Applicant: Field Controls, LLC, Kinston, NC (US)

(72) Inventors: Mark R. LUNDBERG, Greenville, NC (US); Kyle R. Glover, Kinston, NC (US)

(21) Appl. No.: 19/210,417

(22) Filed: May 16, 2025

Related U.S. Application Data
(60) Provisional application No. 63/657,492, filed on Jun. 7, 2024.

(51) Int. Cl. A61L 9/20; F24F 8/22

(52) U.S. Cl. CPC A61L 9/20; F24F 8/22; A61L 2209/11; A61L 2209/15; A61L 2209/16

(57) ABSTRACT

An Ultraviolet (UV) lamp assembly for ducts used in an HVAC system can be mounted onto duct work to facilitate air sanitization. The UV lamp assembly includes a mounting base (attachable to a duct) and a UV lamp powerhead (e.g., with UV bulb) that is removably attachable to the mounting base by rotating the powerhead onto the mounting base. The mounting base can be pre-installed on a duct and capped for subsequent installation of the UV lamp assembly. The UV lamp powerhead can include a switch that powers the UV bulb, and the mounting base can include a switch activator, which is effective to activate the switch at various relative rotated positions of the powerhead. Thus, the switch can be activated (and the lamp powered) when the powerhead is installed at various positions, which can reduce the risk of inadvertent UV lamp activation where the powerhead is not fully rotated.

### CROSS-REFERENCE TO RELATED APPLICATION

This Non-Provisional Patent application claims priority to U.S. Provisional Patent Application No. 63/657,492, filed Jun. 7, 2024, and titled “Ultraviolet (UV) Lamp Assembly”, the entire contents of which is incorporated herein by reference.

### BACKGROUND

Ultraviolet (UV) lamp assemblies typically include a UV blub (e.g., including low-pressure mercury lamps, high-pressure mercury lamps, UV LEDs, fluorescents, HID lamps, etc.) and a housing or enclosure to protect the UV lamp and the internal components of the assembly (e.g., power source, ballast or driver, cooling system, etc.). UV lamp assemblies are used in a wide range of applications, including air and water purification, surface disinfection, curing of coatings and adhesives, sterilization of medical equipment, and more. For example, UV lamp assemblies may be inserted and/or installed into the ducts of heating, ventilation, and air condition (HVAC) systems. In that example, air gets pulled through the duct, sanitized by the rays emitting from the UV lamp, and sent throughout the house. In some cases, the rays emitting from the UV lamp can be damaging to human eyes.

### DETAILED DESCRIPTION OF DRAWINGS

The present systems and methods for an ultraviolet (UV) lamp mounting assembly are described in detail below with reference to these figures.

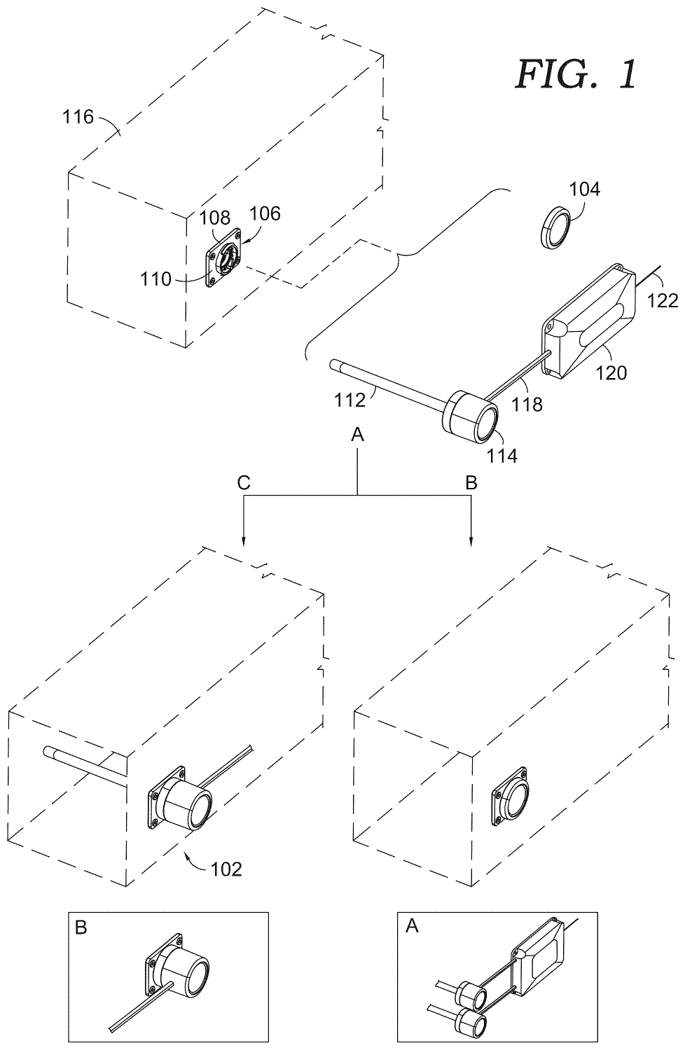

FIG. 1 depicts a UV lamp assembly and a cap, each installed on a mounting base attached to a plenum and/or HVAC system, in accordance with examples of this disclosure.

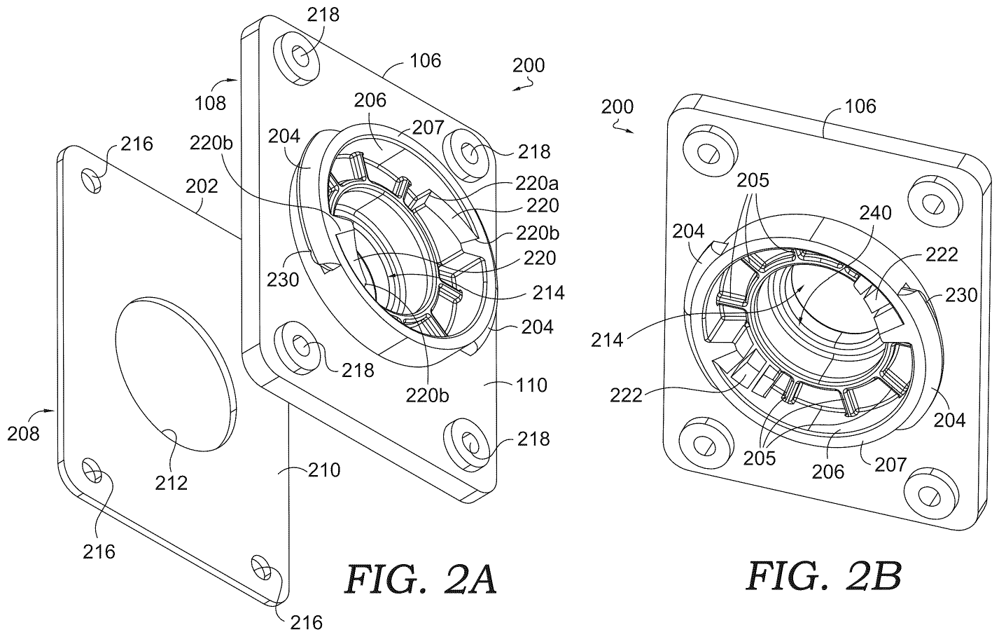

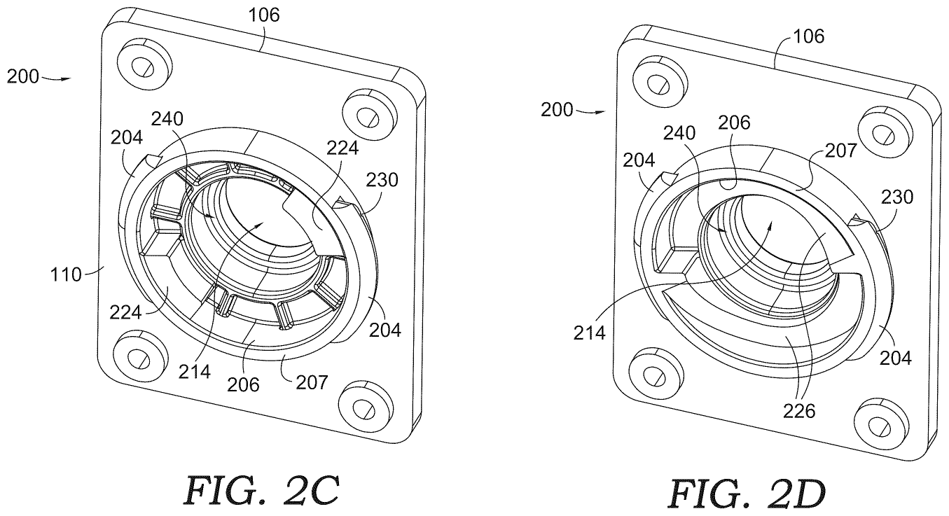

FIG. 2A depicts an exploded view of a mounting base with ramp switch activators, in accordance with examples of this disclosure.

FIG. 2B depicts a mounting base with discontinuously segmented ramp switch activators, in accordance with examples of this disclosure.

FIG. 2C depicts a mounting base with taller switch activators (as compared to FIG. 2A), in accordance with examples of this disclosure.

FIG. 2D depicts a mounting base with long or extended ramp switch activators (as compared to FIG. 2A), in accordance with examples of this disclosure.

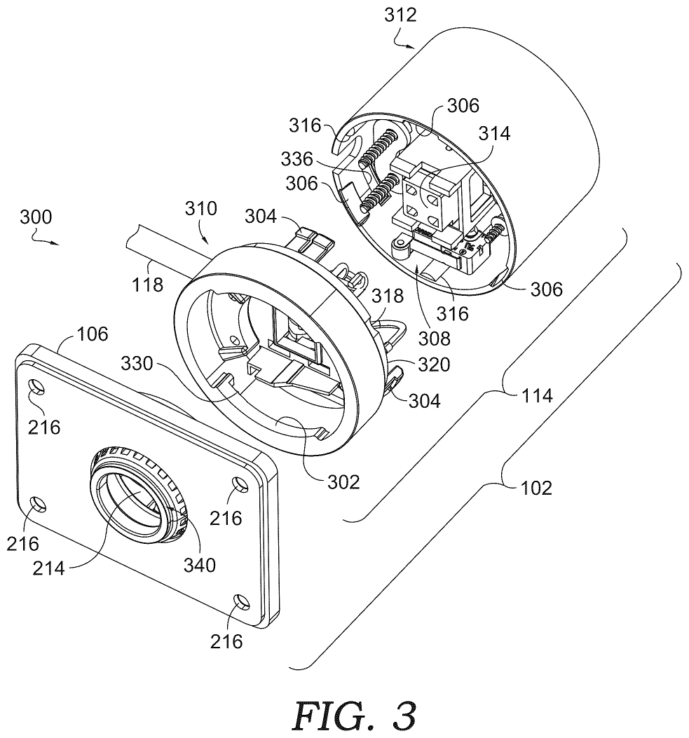

FIG. 3 depicts an exploded view of a UV lamp assembly, in accordance with examples of this disclosure.

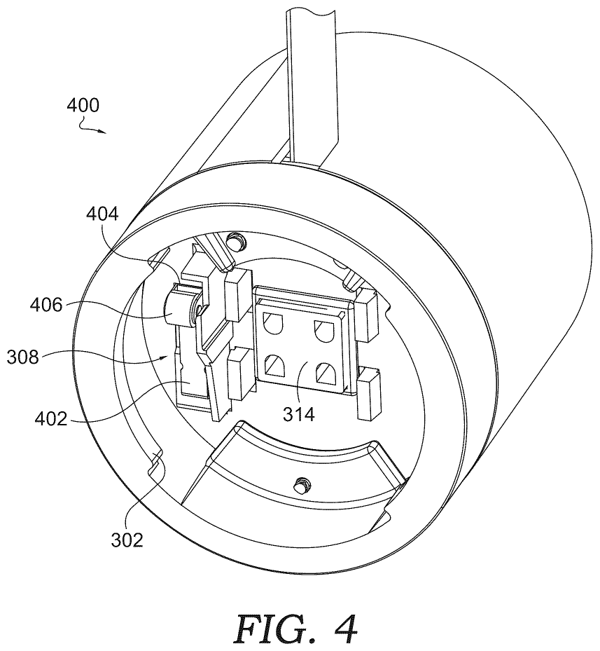

FIG. 4 depicts a microswitch, in accordance with examples of this disclosure.

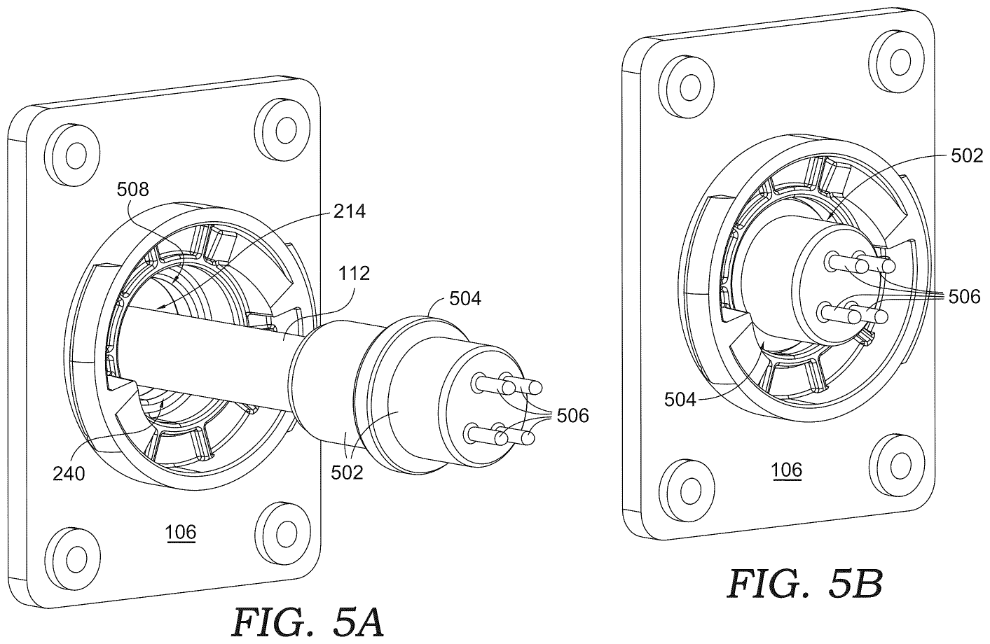

FIG. 5A depicts a UV lamp partially inserted into a mounting base, in accordance with examples of this disclosure.

FIG. 5B depicts a UV lamp fully inserted into a mounting base, in accordance with examples of this disclosure.

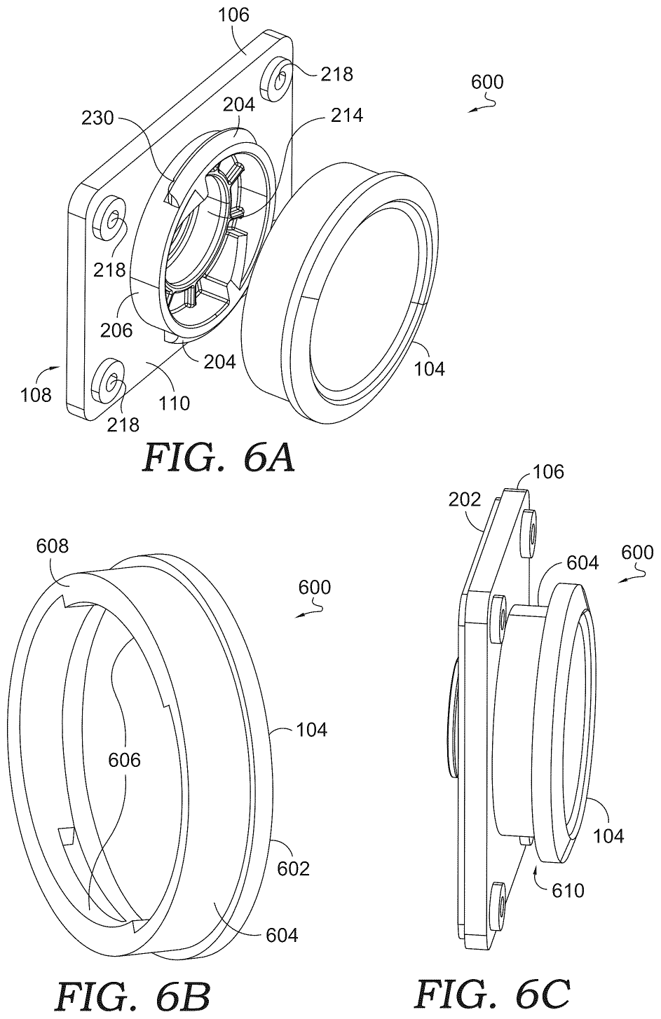

FIG. 6A depicts an exploded view of a mounting base with a cap, in accordance with examples of this disclosure.

FIG. 6B depicts a cap for a mounting base, in accordance with examples of this disclosure.

FIG. 6C depicts a cap attached to a mounting base, in accordance with examples of this disclosure.

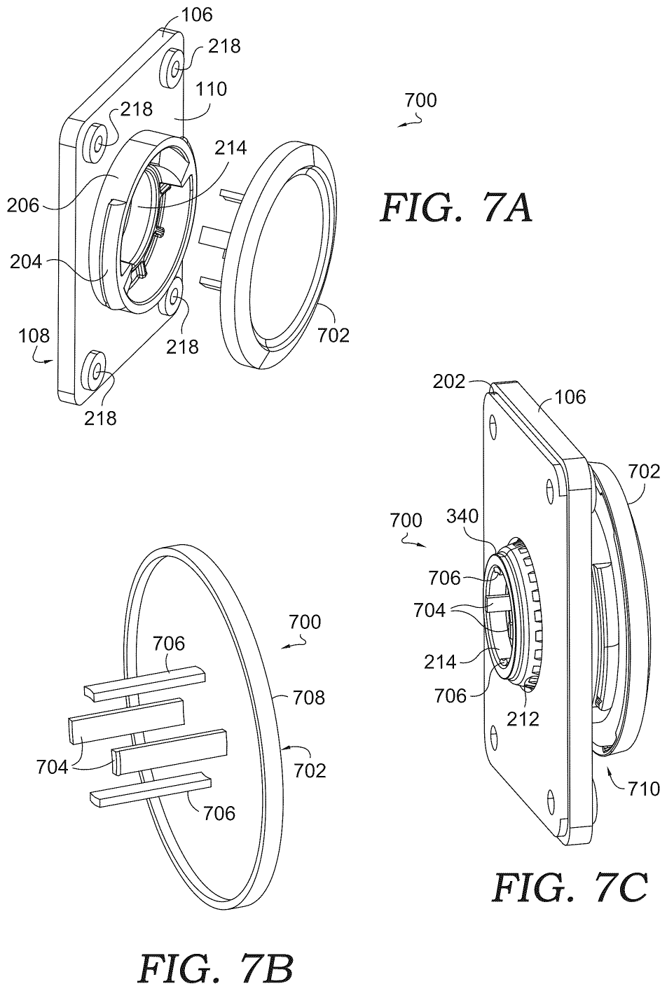

FIG. 7A depicts an exploded view of a mounting base with a plug, in accordance with examples of this disclosure.

FIG. 7B depicts a plug for a mounting base, in accordance with examples of this disclosure.

FIG. 7C depicts a plug attached to a mounting base, in accordance with examples of this disclosure.

### DETAILED DESCRIPTION

This detailed description is related to an Ultraviolet (UV) lamp assembly for any system used to direct air flow (e.g., an air-flow system). For example, the UV lamp assembly can be installed in an HVAC system with a furnace and air conditioner, a fresh-air ventilation system, heat-pump system, and/or any other air-flow system designed to direct air flow. In examples, the UV lamp assembly can be mounted onto duct work of a system (e.g., such as an HVAC system). For example, the UV lamp assembly includes a mounting base (e.g., base plate), which is attachable (e.g., mountable) to a duct, and a UV lamp powerhead (e.g., with UV bulb) that is removably attachable to the mounting base by twisting or rotating the powerhead onto the mounting base. In at least some examples, the mounting base can be pre-installed on a duct (e.g., when the duct is shipped or otherwise before the duct is originally installed) and capped for subsequent installation of the UV lamp (e.g., days, months, years, etc. after the duct is installed). Among other things, pre-installation of the mounting base can facilitate easier, less expensive, and more infallible subsequent UV lamp installation (e.g., since the subsequent UV lamp installation will not require, by someone on-site such as an installation technician or a property owner, cutting a hole in the duct or fastening the mounting base).

In addition, the UV lamp powerhead can include a switch (e.g., microswitch) that powers the UV bulb, and the mounting base can include a switch activator. In some examples, the switch activator on the mounting base is effective to activate the switch in the powerhead even when the powerhead is installed at a number of different orientations (e.g., home positions) relative to the base (e.g., various positions to which the powerhead can be rotated relative to the mounting base). For example, the powerhead can be installed on the mounting base at multiple different orientations (e.g., a first orientation and a second orientation that is 180 degrees relative to the first orientation) and the switch activator on the mounting base can still effectively activate the switch when the powerhead is at any of the multiple different orientations. In at least some examples, the UV lamp assembly can include features that indicate to an installer when the powerhead has been fully rotated to a home position. For example, the powerhead and base can include structures (e.g., protuberances, notches, etc.) that engage one another upon rotation of the powerhead and that produce an audible and/or tactile click. In addition, the structures can help to lock the powerhead in position and reduce the likelihood of inadvertent rotation in the opposite direction.

In contrast to the present invention, conventional UV lamp solutions often lack the effectiveness and durability of the current disclosure. For instance, some conventional UV lamp assemblies can only be assembled in a single orientation (e.g., by placing a powerhead onto a mounting base at a position and rotating the powerhead into the only home position). In another example, conventional UV lamp assemblies can contain a finger (e.g., protruding finger) that extends outward from a wall of the mounting base and that operates to contact a microswitch associated with the UV lamp powerhead when the powerhead is fully rotated. Among other things, the protruding finger can be prone to breaking and can also make it difficult to attach a cap. In addition, conventional solutions (e.g., including a finger) often require the UV lamp powerhead to be fully rotated before the finger contacts the microswitch (e.g., a side of the microswitch). However, if the powerhead is not fully rotated, or if the powerhead rotates back in the opposite direction (e.g., the finger rotates away from the switch), then the UV lamp assembly inadvertently powers off. In other words, the powerhead of conventional solutions must be nearly perfectly rotated as far as possible (e.g., without going too far), in the right direction, and in the right position to cause sufficient contact between the edge of the finger and the microswitch.

If the conventional powerhead rotates (e.g., due to natural vibrations, inclement weather, bumped while children are playing, and/or any other reason that would cause the powerhead to rotate) even slightly in the opposite direction (e.g., away from the edge of the finger), then there would no longer be sufficient contact between the finger and the microswitch (causing inadvertent powering down of the UV system). This can be problematic for several reasons. First, the air moving through the ducts of the air-flow system would not be sanitized since the UV bulb is off (e.g., not emitting UV rays). Second, because users do not often check their UV lamp assemblies to make sure that the powerhead is sufficiently twisted as to engage the finger of the mounting base, the user may endure a substantial period of time until realizing their UV lamp assembly is not operating as intended.

In contrast to conventional solutions, the UV lamp assembly described herein is easily installable and includes robust and reliable microswitch activation. In at least some examples, the mounting base of the present disclosure includes a ramp switch activator that is integrally formed directly into a side wall of the mounting base. As such, in contrast to the finger of some conventional solutions, the ramp switch activator does not include extensions that are relatively easy to break off.

In some examples, the ramp switch activator can include an inclined surface that is positioned to engage the microswitch when the powerhead is engaged to the mounting base. In addition the microswitch can be activated upon contacting various points along the incline, such that the powerhead can be rotated to varying degrees relative to the mounting base and the power source is still activated. Furthermore, in some examples, the mounting base can include a plurality of ramp switch activators, any of which can effectively activate the microswitch, such that the powerhead can be attached at any orientation relative to the mounting base and be operational.

In at least some example, the powerhead can include a more compact form factor, as compared to conventional powerheads, making it easier to manipulate (e.g., twist and/or rotate), less bulky and protruding from the duct upon installation, and easier to ship. In addition, a more compact form allows for multiple powerheads (e.g., an array of powerheads) to be coupled to a duct (or other plenum or HVAC air handler system) in the same general region, whereas with conventional, larger powerheads, it can be more challenging to fit multiple powerheads into the same footprint.

In some examples, the powerhead can be more compact, as compared to conventional powerheads, because electrical components that are normally located in a conventional powerhead are enclosed in a separate power assembly. This power assembly may connect to a power source (e.g., a wall socket) to provide power to the powerhead. The benefits of this design include the reduction in size of the powerhead (e.g., as compared to conventional powerheads), and the new design allows for increased power capabilities, which is achieved by the power assembly. As such, higher amounts of power can be pushed through the power assembly. Because more power can be pushed through the power assembly (e.g., as compared to power being conventionally pushed directly to a powerhead from, for example, a wall socket), additional powerheads can be attached to the power assembly. Therefore, multiple powerheads can be positioned in relatively close positions along an air-flow system to more effectively and efficiently sanitize the air moving about the system. In addition, the power assembly allows for greater power to be supplied to larger, more powerful bulbs, thereby requiring fewer bulbs altogether (e.g., fewer bulbs throughout the system, but still achieving desired sanitization levels). For example, a bulb of the present disclosure can be as long as 22 inches, if not greater, due to the greater power harnessed by the power assembly. In contrast, conventional solutions often include shorter length bulbs due to less available power.

FIG. 1 is a state diagram illustrating subject matter of the present disclosure in alternative states. For example, subject matter of the present disclosure is related to a UV lamp assembly 102 having a mounting base 106 and a powerhead 114. In at least some examples, the UV lamp assembly 102 is attachable to a power assembly 120 via a power cable 118 (e.g., a power cord), and the power assembly 120 is connectable to a power source (e.g., such as a wall socket or any other isolated power source) via power line 122.

In some examples, electrical components (e.g., at the end of the power cable 118) engage the power assembly 120 to supply power to the powerhead 114. The power assembly 120 can be pre-configured to receive one or more powerheads 114 (e.g., multiple powerheads 114 can be electrically connected to the power assembly inside the junction box). In some examples, due to the power assembly 120, multiple powerheads 114 can be positioned in close proximity to one another (e.g., as compared to conventional UV lamp assemblies) along an air-flow system (e.g., an HVAC system) to allow for greater air-flow sanitization and/or evaporator coil sanitization (e.g., as compared to conventional UV lamp assemblies). For example, while conventional powerheads are typically capable of supplying a range of power between 220-400 milliamps, the present solution, with the addition of the power assembly 120 sending power to the powerhead 114, is capable of supplying a range of power anywhere from 100-800 milliamps. As such, the present solution supplies more power to the UV lamp assembly 102 than conventional solutions, which means that fewer UV lamp assemblies 102 are required to achieve the same level of sanitization within an air-flow system (e.g., as compared to conventional UV lamp assemblies).

Notably, the UV lamp assembly 102 can be installed in any system used to direct air flow. For example, the UV lamp assembly 102 can be installed in an HVAC system, a fresh-air ventilation system, a fan cooling unit, a fan cooling/heating unit, a heat-pump system, and/or any other air-flow system designed to direct air flow. In an HVAC system, for example, the UV lamp assembly 102 can be installed on the ducts (e.g., sheet metal ducts, flexible ducts, fiberboard ducts, duct board panels, phenolic foam ducts, etc.), before a blower (e.g., centrifugal blower, axial flow fan, mixed flow fan, propeller fan, cross-flow fan, regenerative blower, etc.), after the blower, behind an input, before an output, near an evaporator coil (e.g., A-coil, W or M coil, 3-plate coil, etc.), within an evaporator coil, near an HVAC cabinet (such as on an outer wall of the HVAC cabinet), and/or positioned anywhere in the air-flow system (such as on a duct wall). Furthermore, in contrast to conventional UV lamp assemblies, which are often too big to fit into certain areas along an air-flow system, the UV lamp assembly 102 is compact enough, due to the addition of the power assembly 120, to position one or more powerheads 114 in more areas (e.g., at different points) of the air-flow system. For example, as illustrated in Reference Box A, two powerheads 114 can be positioned side-by-side in an air-flow system, because there is enough space on a plenum to do so (and the more compact size of the powerheads 114 permits them to fit in that space). As such, powerheads 114 can be mounted parallel in circuit.

In some examples, the UV lamp assembly 102 is attachable to a plenum 116 by way of the mounting base 106, which is more permanently installed on the plenum 116 (e.g., via hardware fasteners), and the powerhead 114 that is releasably attachable to the mounting base 106. State “A” in FIG. 1 shows the mounting base 106 attached to the plenum 116 (e.g., duct, as illustrated in the example embodiment depicted in FIG. 1). The plenum 116 can be any space in which a UV lamp assembly 102 is attachable (e.g., an HVAC system, a fresh-air ventilation system, a fan cooling unit, a fan cooling/heating unit, a heat-pump system, and/or any other system designed to direct air flow). In examples, the powerhead 114 can be releasably attached at different orientations relative to the mounting base 106. For example, while state “C” depicts the UV lamp assembly 102 positioned with the power cable 118 pointing to the right, the Reference Box B illustrates another example position in which the UV lamp assembly 102 is positioned with the power cable 118 pointing to the left. In at least some examples, a cap 104 can be releasably coupled to the mounting base, as indicated in state “B” (e.g., prior to installation of the duct and/or after installation of the duct and prior to installation of a powerhead, such as the powerhead 114).

With reference now to FIGS. 2A-2D, FIGS. 2A-2D include depictions of various embodiments of mounting base 106. For example, FIG. 2A depicts an exploded view of an example mounting base 106. In some examples, the mounting base 106 includes a gasket 202. In the embodiment depicted in FIG. 2A, the gasket 202 includes gasket fastener holes 216, a first through-hole 212, a back face 208, and a front face 210. The mounting base 106 may be positioned on top of the gasket 202 such that the front face 210 of the gasket 202 is flush against a back face 108 of the mounting base 106.

In some examples, the mounting base 106 may include mounting base fastener holes 218 and a second through-hole 214. A UV bulb 112 (e.g., low-pressure mercury lamps, high-pressure mercury lamps, UV LEDs, fluorescents, HID lamps, etc.) can be inserted through the second through-hole 214 for positioning in the enclosed volume of the plenum 116. The second through-hole 214 includes a tapered end 240 (e.g., tapers from a wider opening closer to the front face 110 to a narrower opening closer to the back face 108) to help center the UV bulb 112 when the UV bulb 112 is inserted into the second through-hole 214.

The mounting base fastener holes 218 and the second through-hole 214 are configured to align with the gasket fastener holes 216 and the first through-hole 212, respectively. In some examples, the back face 208 of gasket 202 may be positioned against the plenum 116 such that the second through-hole 214 of mounting base 106 leads into the first through-hole 212 of gasket 202, which leads into a through-hole (not pictured) of the plenum 116. When the three through holes are aligned (e.g., the first through-hole 212, the second through-hole 214, and a through-hole on a side of the plenum 116), and the mounting base fastener holes 218 are aligned with the gasket fastener holes 216, then the mounting base 106 (and the gasket 202) may be secured to a side of the plenum 116 with some type of fastener (e.g., screws, bolts, lags, nails, rivets, etc.) by inserting the fastener through each fastener hole. Furthermore, a collar end (e.g., see collar end 340 of FIG. 3), which defines the end of the second through-hole 214, of the mounting base 106 includes a groove that is configured to mate with a portion of the plenum 116 that defines the through-hole (not pictured) of the plenum 116. Therefore, in at least some examples, the mounting base 106 is securable to the plenum 116 (e.g., ductwork). In at least some examples, the mounting base 106 may be pre-installed on a side of the plenum 116 (e.g., ductwork), and a cap 104 may be used to cover the opening (e.g., second through-hole 214) of the mounting base 106.

In some examples, the mounting base 106 may also include a wall 206 (e.g., an externally threaded portion of the opening) that protrudes from a front face 110 of mounting base 106 and radially around the second through-hole 214. In some examples, at least one external mounting flange 204 extends outward from the external side of the (e.g., radial) wall 206. In at least some examples, the wall 206 is supported by at least one rib 205 (e.g., called out in FIG. 2B). These ribs 205 strengthen the durability of the wall 206 and prevent warping of the wall 206.

FIGS. 2A-2D each depict different example configurations of the internal side of wall 206. For example, each of FIGS. 2A-2D depicts one of a plurality of (e.g., ramp) switch activators positioned along the internal side of wall 206. In at least some examples, a switch activator is operational to engage a microswitch that is positioned on the inside of the powerhead 114.

For example, FIG. 2A depicts two ramp switch activators 220, and there could be fewer ramp switch activators or more ramp switch activators. In this example, both ramp switch activators 220 extend from a first point 220a that is lower to a second point 220b that is higher (e.g., relative to the overall height of the wall 206). In some examples, the first point 220a is positioned about halfway between the front face 110 to a top 207 of the wall 206. In some examples, the ramp switch activator 220 comprises an inclined surface, and the second point 220b can be substantially flush with the top 207 of the wall 206. In some examples, the second point 220b can be lower than the top 207 of the wall 206. In some examples, the second point 220b can be above the top 207 of the wall 206. In some examples, the ramp switch activator 220 partially circumscribes the second through-hole 214 by at least 20 degrees (e.g., as a measure of an arc length or other length associated with the ramp switch activator). In some examples, the ramp switch activator 220 can radially extend around the through-hole by a larger amount, such as in a range between 20 degrees and 180 degrees. In some examples, the ramp switch activator can include a single ramp extending more than 180 degrees radially around the through hole (e.g., the second through-hole 214).

Another configuration of a switch activator can be seen in FIG. 2B. In the example embodiment depicted in FIG. 2B, two discontinuously segmented ramp switch activators 222 are depicted. Similar to the two ramp switch activators 220, both segmented ramp switch activators 222 begin at a point alongside the wall 206 that is lower than the top 207 of the wall 206 (e.g., at least 20 degrees lower). Each segmented ramp switch activator 222 comprises one or more discontinuous ramp segments. In the example embodiment depicted in FIG. 2B, each segmented ramp switch activator 222 includes three separate segments. However, the segmented ramp switch activators 222 may include any number of separate segments.

In yet another example embodiment, FIG. 2C depicts two taller and flatter switch activators 224 (as compared to the switch activator 220). The flatter switch activators 224 extend from the inner side of wall 206 and are flush with the top 207 of wall 206 or extend above the top 207 of the wall 206. In some examples, the flatter switch activators 224 extend from the inner side of wall 206 and extend below the top 207 of the wall 206. In some examples, the switch activators 224 are substantially flat or can have a slight incline.

Furthermore, FIG. 2D depicts another example embodiment of a switch activator. As depicted in the example illustrated in FIG. 2D, the switch activator may be a long or extended ramp switch activator 226. Each long or extended ramp switch activator 226 begins at the base of an adjacent extended switch activator 226 and rises to the top 207 of wall 206. In some examples, the extended ramp switch activator 226 rises to a point below the top 207 of wall 206.

Although many of the example embodiments of switch activators depicted in FIGS. 2A-2D contain two switch activators (e.g., two ramp switch activators 220, two segmented ramp switch activators 222, two flatter switch activators 224, and two extended switch activators 226), some examples can include a single switch activator. However, the present disclosure contemplates any number of switch activators (e.g., 1, 2, 3, 4, etc.).

Referring now to FIG. 3, FIG. 3 depicts an exploded view of the UV lamp assembly 102. In some examples, the UV lamp assembly 102 includes the mounting base 106 and the powerhead 114. The powerhead 114 includes a first portion 310 for attaching to the mounting base 106 and a second portion 312 for housing electrical components, such as a UV bulb socket 314 (e.g., where an end of the UV bulb 112 is inserted into), and other components of the powerhead 114. In some examples, clips 304 on the first portion 310 of the powerhead 114 connect to (e.g., mate with) grooves 306 and recessed mating indents 336 in the second portion 312 of the powerhead 114, which operatively attaches the first and second portions to form the powerhead 114. In at least some examples, half-moon protuberances 316 (e.g., locating structures), located radially and internally within the second portion 312, are configured to mate with support recesses 318, located on an outer ring 320 at an end of the first portion 310 (e.g., the end of the first portion 310 configured to mate with an end of the second portion 312). The half-moon protuberances 316 are configured to mate with the support recesses 318 such that the mating removes stress from the clips 304 (e.g., when connected to the grooves 306 and the recessed mating indents 336) associated with rotational (e.g., twist) force, which helps to prevent breakage and component separation (e.g., separation of the first portion 310 and the second portion 312).

Furthermore, the first portion 310 is connected to the power cable 118, which leads to the power assembly 120. In some examples, the microswitch 308 is operatively connected to the power cable 118. In some examples, the microswitch 308 is housed within the powerhead 114, and in FIG. 3, a general position of the microswitch 308 is identified, while FIG. 4 shows a different view of the microswitch 308.

The powerhead 114 is attachable to the mounting base 106 via at least one connection lip 302 (e.g., an edge or thread-like part of the internal groove/recess of the powerhead 114). For example, the mounting base 106 is attachable, via external mounting flanges 204 that are external relative to the wall 206 (e.g., see FIGS. 2A-2D), to the at least one connection lip 302 of the powerhead 114 (e.g., power source, such as a ballast) of the UV lamp assembly 102. In at least some embodiments, the powerhead 114 of the UV lamp assembly 102 is attachable to the mounting base 106 (e.g., at any of multiple positions) by twisting or rotating the at least one connection lip 302 of the powerhead 114 onto the external mounting flanges 204 (e.g., ears, tabs, prongs, etc.) of the wall 206.

Furthermore, in some examples, a notch 230 on the exterior mounting flanges 204 (e.g., see FIGS. 2A-2D) mates with a protuberance 330 located on the at least one connection lip 302, which may indicate to an installer that the powerhead has been fully rotated to a home position. For example, when the powerhead 114 is rotated to a home position, the notch 230 engages (e.g., clicks into) the protuberance 330, which produces an audible and/or tactile click. In addition, these structures (e.g., the notch 230 and the protuberance 330) can help to lock the powerhead in position and reduce the likelihood off inadvertent rotation in the opposite direction. Accordingly, the connection lip 302 is configured to releasably mate with the external mounting flanges 204. As such, the microswitch 308 is positioned to engage the switch activator (e.g., ramp switch activator 220, segmented ramp switch activator 222, flatter switch activator 224, and/or extended switch activator 226) when the connection lip 302 is mated with the external mounting flange 204.

With reference now to FIG. 4, FIG. 4 depicts the components of the microswitch 308. In some examples, the microswitch 308 includes a lever 402, a distal end 404 of the lever 402, and a roller 406. In some examples, the microswitch 308 may not have a roller 406 (e.g., wheel end), but rather can have a flat end or a formed end (e.g., not pictured in the example illustrated in FIG. 4). The microswitch 308 is the electrical component that opens or closes a circuit (e.g., powering the UV lamp assembly 102). The lever 402 is the trigger that operates the microswitch (e.g., closes or opens the circuit). In some examples, the roller 406 is configured to roll along one of the switch activator configurations (e.g., ramp switch activator 220, segmented ramp switch activator 222, flatter switch activator 224, and/or extended switch activator 226), which causes the distal end 404 of lever 402 to activate the microswitch 308 and close the circuit, powering the UV lamp assembly 102.

In some examples, the switch activator is effective to activate the microswitch 308 at various relative rotated positions of the powerhead 114 (e.g., various positions to which the powerhead 114 can be rotated relative to the mounting base 106). As such, the microswitch 308 can be activated (and the UV lamp assembly 102 powered) when the powerhead 114 is installed at various positions (e.g., twisting the at least one connection lip 302 onto the external mounting flange 204), which can reduce the risk of inadvertent UV lamp assembly 102 activation where the powerhead 114 is not fully rotated. Furthermore, in at least some examples, the powerhead 114 can be installed by rotating the powerhead 114 onto the mounting base 106 in the clockwise direction. For example, the powerhead 114 can be installed on the mounting base 106 and activate the microswitch 308 by rotating the powerhead 114 by 90 degrees onto the mounting base 106 in the clockwise direction (e.g., a universal orientation). Accordingly, the position in which the powerhead 114 is attached to the mounting base 106 does not matter, and the direction in which the powerhead 114 is attached to the mounting base 106 also does not carry the same consequences as that of conventional solutions. For example, regarding the present disclosure, twisting (e.g., rotating) the powerhead 114 onto the mounting base 106 does not carry the risk of breaking a finger. This is in contrast to a conventional UV lamp assembly, which could result in a broken finger if the powerhead is not properly attached to, or inserted into, a conventional mounting base (e.g., such a broken finger could render the conventional UV lamp assembly inoperable).

As such, it is less critical at which point along a radial relative position that the powerhead 114 is attached to the mounting base 106, because twisting the powerhead 114 along the external mounting flanges 204 will attach the powerhead 114 onto the mounting base 106, engage the microswitch 308 with the switch activator (e.g., ramp switch activator 220, segmented ramp switch activator 222, flatter switch activator 224, and/or extended switch activator 226), and turn on the UV bulb 112 inside the plenum 116, without any potentially harmful UV rays escaping outside of the plenum 116. For at least these reasons, the UV lamp assembly 102 disclosed herein tends to be more effective (e.g., activates more consistently) and durable (e.g., less prone to breaking or improper installment) than conventional solutions.

Referring now to FIGS. 5A and 5B, the second through-hole 214 is configured to secure the UV bulb 112 when the powerhead 114 is attached to the mounting base 106. As an installer (e.g., a person installing the UV lamp assembly 102) moves the UV bulb 112 into the second through-hole 214 (e.g., moving the powerhead 114 closer to the mounting base 106), the UV bulb 112 approaches the position it will be in once the UV lamp assembly 102 is installed. In at least some examples, the UV bulb 112 includes a sleeve 502, a sleeve collar 504, and metal prongs 506 (e.g., four metal prongs are depicted in this embodiment, but any number of prongs is contemplated). The metal prongs 506 can be inserted into the UV bulb socket 314 (e.g., four openings are depicted in FIGS. 3 and 4, but any number of openings in the UV bulb socket 314 that match the metal prongs 506 are contemplated), thereby providing power to the UV bulb 112 and illuminating the bulb when the microswitch 308 is activated. In at least some examples, the tapered end 240 of the second through-hole 214 helps to center the UV bulb 112 when it is inserted into the second through-hole 214. For example, the bottom portion of the sleeve 502 abuts against the tapered end 240, causing friction that helps to hold the UV bulb 112 in place. Furthermore, a through-hole flange 508, located near the tapered end 240, is configured so that the sleeve collar 504 rests flush on top of the through-hole flange 508 (e.g., as depicted in FIG. 5B) when the powerhead 114 is attached to the mounting base 106.

In some examples, it may be desirable to install the powerhead 114 at a date later than the installation of plenum 116. In contemplation of that scenario, the mounting base 106 can be pre-installed on a plenum 116 (e.g., when the duct is shipped or otherwise before the duct is originally installed) and capped for subsequent installation of the powerhead 114 (e.g., days, months, years, etc. after the duct is installed) to form the UV lamp assembly 102. Among other things, pre-installation of the mounting base 106 can facilitate easier, less expensive, and more infallible subsequent UV lamp assembly 102 installation (e.g., since the subsequent UV lamp assembly 102 installation will not require, by someone on-site such as an installation technician or a property owner, cutting a hole in the duct or fastening the mounting base).

Referring now to FIGS. 6A-6C, FIGS. 6A-6C depict the cap 104, which is configured to attach to the mounting base 106 and that covers the second through-hole 214 of mounting base 106 once attached. As illustrated in FIGS. 6A-6C, the cap 104 is attachable to the one or more eternal mounting flanges 204. In some examples, the cap 104 includes dimensions that contribute to a relatively low profile when pre-installed on the duct (e.g., the cap 104 might protrude from the front face 110 by less than an inch or lower).

With reference now to FIG. 6B, FIG. 6B depicts the cap 104 with a cap top 602, a ring 608, a perimeter wall 604, and at least one twist lock flange 606. The cap top 602 covers the second through-hole 214. The ring 608 of the cap 104 (e.g., comprising the perimeter wall 604) fits around the wall 206 of the mounting base 106 once the cap 104 is attached to the mounting base 106. In some examples, the mounting base 106 is attachable, via external mounting flanges 204, to the twist lock flanges 606 of the cap 104. In at least some embodiments, the cap 104 is attachable to the mounting base 106 by twisting or rotating the at least one twist lock flange 606 onto the external mounting flanges. As such, the cap 104 is configured to releasably mate with the external mounting flanges 204.

Referring now to FIG. 6C, FIG. 6C depicts an attached cap 610. In some examples, the cap 104 attaches to the front face 110 of the mounting base 106 and is offset by some distance that is less than an attached powerhead 114. In some embodiments, the cap 104 fits flush against the terminal portion of the wall 206 that is distal relative to the front face 110 of the mounting base 106. In some examples, covering the second through-hole 214 of the mounting base 106 with the cap 104 is beneficial, because the cap 104, while lacking air sanitization capabilities (e.g., such as the UV lamp assembly 102), keeps air within the air-flow system from escaping through the second through-hole 214.

Referring now to FIGS. 7A-7C, FIGS. 7A-7C depict a plug cap 702, which is configured to attach to the mounting base 106 and that covers the second through-hole 214 of mounting base 106 once attached. As illustrated in FIGS. 7A-7C, the plug cap 702 can be inserted into the second through 214 of mounting base 106.

With reference now to FIG. 7B, FIG. 7B depicts the plug cap 702 with two undercut prongs 704 (e.g., with barb-like hooks or protrusions at the end), two straight prongs 706, and a plug cap top 708. The plug cap top 708 can fit flush against the terminal portion of the wall 206 that is distal relative to the front face 110 of the mounting base 106. The plug cap 702 covers the second through-hole 214 when it is inserted into the second through-hole 214. In some examples, the undercut prongs 704 latch on to the bottom of the second through-hole 214, positioning the plug cap 702 in an attached position. Furthermore, the straight prongs 706 push against the inner wall of the second through-hole 214, helping to keep the plug cap 702 in place. A user may remove the plug cap 702 by applying a certain amount of force in the direction opposite of the front face 110 of the mounting base 106. As such, the plug cap 702 is configured to releasably mate with the second through-hole 214.

Referring now to FIG. 7C, FIG. 7C depicts an attached plug cap 710. In some examples, the plug cap 702 is inserted into the second through-hole 214, and the second through-hole 214 is inserted into the first through-hole 212. In some examples, covering the second through-hole 214 of the mounting base 106 with the plug cap 702 is beneficial, because the plug cap 702, while lacking air sanitization capabilities (e.g., such as the UV lamp assembly 102), keeps air within the air-flow system from escaping through the second through-hole 214.

### Example Clauses

A. A UV lamp assembly comprising: a mounting base, which is configured to attach to a plenum, and a powerhead that includes a UV bulb and that releasably attaches to the mounting base; the mounting base comprising: a plate comprising a back face, which is configured to be oriented towards the plenum, and a front face that, relative to the back face, is on an opposite side of the plate; a through-hole extending from the front face to the back face and configured to receive the UV bulb; a wall that extends from the front face and that includes an internal side towards the through-hole and an external side away from the through-hole; an external mounting flange positioned on the external side of the wall; and a ramp positioned on the internal side of the wall; and the powerhead comprising: a connection lip configured to releasably mate with the external mounting flange; and a microswitch positioned to engage the ramp when the connection lip is mated with the external mounting flange.

B. The UV lamp assembly of Clause A, further comprising a cap configured to releasably attach to the mounting base when the powerhead is not attached to the mounting base.

C. The UV lamp assembly of Clauses A and B, further comprising one or more additional powerheads connected to a power assembly.

D. The UV lamp assembly of Clauses A-C, wherein the powerhead and the one or more additional powerheads are mounted to the plenum and parallel in a circuit.

E. The UV lamp assembly of Clauses A-D, wherein the ramp comprises a first ramp and the mounting base comprises at least a second ramp.

F. The UV lamp assembly of Clauses A-E, wherein the ramp comprises one or more discontinuous ramp segments.

G. The UV lamp assembly of Clauses A-F, wherein the ramp comprises an inclined surface, and wherein at least a portion of the inclined surface extends beyond a terminal portion of the wall that is distal relative to the front face.

H. The UV lamp assembly of Clauses A-G, wherein the ramp at least partially circumscribes the through-hole by at least 20 degrees.

I. The UV lamp assembly of Clauses A-H, wherein the ramp comprises a flat top that is flush with a terminal portion of the wall that is distal relative to the front face.

J. The UV lamp assembly of Clauses A-I, wherein the ramp comprises an inclined surface, and wherein the microswitch is configured to engage the ramp at multiple points along the inclined surface.

K. The UV lamp assembly of Clauses A-J, wherein the microswitch engaging the ramp at any of the multiple points activates the microswitch.

L. The UV lamp assembly of Clauses A-K, wherein the microswitch comprises a lever with a distal end configured to traverse along the ramp.

M. The UV lamp assembly of Clauses A-L, wherein the distal end comprises a roller.

N. The UV lamp assembly of Clauses A-M, wherein the microswitch is activated when the distal end rolls up the ramp and at any of the multiple points.

O. The UV lamp assembly of Clause A-N, wherein the UV lamp assembly is installed before a blower of an air-flow system.

P. The UV lamp assembly of Clauses A-O, wherein the UV lamp assembly is installed after a blower of an air-flow system.

Q. The UV lamp assembly of Clauses A-P, wherein the UV lamp assembly is installed in front of an input of an air-flow system.

R. The UV lamp assembly of Clauses A-Q, wherein the UV lamp assembly is installed behind an input of an air-flow system.

S. The UV lamp assembly of Clauses A-R, wherein the UV lamp assembly is installed near an evaporator coil of an air-flow system.

T. The UV lamp assembly of Clauses A-S, wherein the UV lamp assembly is installed within an evaporator coil of an air-flow system.

U. The UV lamp assembly of Clauses A-T, wherein the UV lamp assembly is installed near an HVAC cabinet of an HVAC system.

V. A UV lamp assembly comprising: a powerhead comprising: a power cable; a microswitch operatively connected to the power cable; and a UV bulb socket; and a mounting base that is releasably connectable to the powerhead, the mounting base comprising a ramp that activates the microswitch when the powerhead is connected to the mounting base.

W. The UV lamp assembly of Clause V, wherein the ramp comprises an inclined surface, and wherein the microswitch is configured to engage the ramp at multiple points along the inclined surface.

X. The UV lamp assembly of Clauses V and W, wherein the microswitch engaging the ramp at any of the multiple points activates the microswitch.

Y. The UV lamp assembly of Clauses V-X, wherein the microswitch comprises a lever with a distal end configured to traverse along the ramp.

Z. The UV lamp assembly of Clauses V-Y, wherein the distal end comprises a roller.

AA. The UV lamp assembly of Clauses V-Z, wherein the microswitch is activated when the distal end rolls up the ramp and at any of the multiple points.

BB. A UV lamp pre-assembly comprising: an HVAC system comprising a plenum at least partially enclosed by a wall, wherein the wall comprises a first through-hole; a mounting base that is coupled to the wall and that comprises: a plate comprising a back face, which is oriented towards the wall, and a front face that, relative to the back face, is on an opposite side of the plate; and a second through-hole extending from the front face to the back face and at least partially aligned with the first through-hole; and a cap that is attached to the mounting base and that covers the second through-hole.

CC. The UV lamp pre-assembly of Clause BB, further comprising a gasket that has a front face, which is oriented towards the plate, and a back face, relative to the front face, is on an opposite side of the plate.

DD. The UV lamp pre-assembly of Clauses BB and CC, wherein the gasket comprises a third through-hole.

EE. The UV lamp pre-assembly of Clauses BB-DD, wherein the first through-hole, the second through-hole, and the third through-hole are aligned.

FF. The UV lamp pre-assembly of Clauses BB-EE, further comprising: the back face of the plate positioned against the front face of the gasket; the back face of the gasket positioned against the wall; and at least one fastener connecting the plate, the gasket, and the wall.

GG. The UV lamp pre-assembly of Clauses BB-FF, wherein a wall that extends from the second through-hole and includes an internal side towards the second through-hole and an external side away from the second through-hole.

HH. The UV lamp pre-assembly of Clauses BB-GG, wherein the external side of the wall comprises one or more mounting flanges that are externally threaded.

II. The UV lamp pre-assembly of Clauses BB-HH, wherein the cap is attachable to the one or more mounting flanges.

JJ. The UV lamp pre-assembly of Clauses BB-II, wherein the internal side wall comprises a ramp.

KK. The UV lamp pre-assembly of Clauses BB-JJ, wherein the ramp comprises one or more discontinuous ramp segments.

LL. The UV lamp pre-assembly of Clauses BB-KK, wherein the ramp comprises an inclined surface, and wherein at least a portion of the inclined surface extends beyond a terminal portion of the wall that is distal relative to the front face.

MM. The UV lamp pre-assembly of Clauses BB-LL, wherein the ramp at least partially circumscribes the through-hole by at least 20 degrees.

NN. The UV lamp pre-assembly of Clauses BB-MM, wherein the ramp comprises a flat top that is flush with a terminal portion of the wall that is distal relative to the front face.

OO. The UV lamp pre-assembly of Clauses BB-NN, wherein the ramp comprises a first ramp and the cap comprises at least a second ramp.

PP. The UV lamp pre-assembly of Clauses BB-OO, wherein the ramp comprises an inclined surface, and wherein the cap is configured to engage the ramp at multiple points along the inclined surface.

QQ. The UV lamp pre-assembly of Clauses BB-PP, wherein the cap comprises a height that is equal to or less than 1 inch.

RR. The UV lamp pre-assembly of Clauses BB-QQ, wherein the cap is attached on the front face and is offset by some distance that is less than an attached powerhead.

SS. The UV lamp pre-assembly of Clauses BB-RR, wherein the cap sits flush on the mounting base

TT. The UV lamp pre-assembly of Clauses BB-SS, wherein the cap comprises a plug.

UU. The UV lamp pre-assembly of Clauses BB-TT, wherein the plug comprises at least two undercut ribs and is insertable into the second through-hole, wherein the at least two undercut ribs are attachable to a bottom of the second through-hole.

VV. The UV lamp pre-assembly of Clauses BB-UU, wherein the wall is an outer wall of an HVAC cabinet.

WW. The UV lamp pre-assembly of Clauses BB-VV, wherein the wall is a duct wall.

XX. A method of providing a UV lamp assembly for a plenum of an air-flow system, the method comprising: attaching, prior to installing the plenum in the air-flow system, a mounting base to a wall of the plenum; attaching a cap to the mounting base; and installing, with the mounting base and the cap pre-installed, the plenum in the air-flow system.

YY. The method of Clause XX, further comprising, subsequent to installing the plenum, removing the cap and attaching, to the mounting base, a powerhead with a UV bulb.

As used herein, a recitation of “and/or” with respect to two or more elements should be interpreted to mean only one element, or a combination of elements. For example, “element A, element B, and/or element C” may include only element A, only element B, only element C, element A and element B, element A and element C, element B and element C, or elements A, B, and C. In addition, “at least one of element A or element B” may include at least one of element A, at least one of element B, or at least one of element A and at least one of element B. Further, “at least one of element A and element B” may include at least one of element A, at least one of element B, or at least one of element A and at least one of element B.

The present embodiments are described with reference to the drawings in which like elements are referred to by like numerals. The relationship and functioning of the various elements of this disclosure are better understood from the detailed description. However, the embodiments of the disclosure are not limited to the embodiments illustrated in the drawings. It should be understood that in certain instances, details have been omitted which are not necessary for an understanding of the present disclosure, such as conventional fabrication and assembly.

This detailed description is provided in order to meet statutory requirements. However, this description is not intended to limit the scope of the invention described herein. Rather, the claimed subject matter may be embodied in different ways, to include different steps, different combinations of steps, different elements, and/or different combinations of elements, similar or equivalent to those described in this disclosure, and in conjunction with other present or future technologies. The examples herein are intended in all respects to be illustrative rather than restrictive. In this sense, alternative examples or examples can become apparent to those of ordinary skill in the art to which the present subject matter pertains without departing from the scope hereof.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

Claimed is:

1. A UV lamp assembly comprising:
a mounting base, which is configured to attach to a plenum, and a powerhead that includes a UV bulb and that releasably attaches to the mounting base;
the mounting base comprising:
a plate comprising a back face, which is configured to be oriented towards the plenum, and a front face that, relative to the back face, is on an opposite side of the plate;
a through-hole extending from the front face to the back face and configured to receive the UV bulb;
a wall that extends from the front face and that includes an internal side towards the through-hole and an external side away from the through-hole;
an external mounting flange positioned on the external side of the wall; and
a ramp positioned on the internal side of the wall; and
the powerhead comprising:
a connection lip configured to releasably mate with the external mounting flange; and
a microswitch positioned to engage the ramp when the connection lip is mated with the external mounting flange.

2. The UV lamp assembly of claim 1, further comprising a cap configured to releasably attach to the mounting base when the powerhead is not attached to the mounting base.

3. The UV lamp assembly of claim 1, further comprising one or more additional powerheads connected to a power assembly.

4. The UV lamp assembly of claim 1, wherein the ramp comprises a first ramp and the mounting base comprises at least a second ramp.

5. The UV lamp assembly of claim 1, wherein the ramp comprises a flat top that is flush with a terminal portion of the wall that is distal relative to the front face.

6. The UV lamp assembly of claim 1, wherein the ramp comprises an inclined surface, and wherein the microswitch is configured to engage the ramp at multiple points along the inclined surface.

7. The UV lamp assembly of claim 6, wherein the microswitch comprises a lever with a distal end configured to traverse along the ramp.

8. The UV lamp assembly of claim 7, wherein the distal end comprises a roller.

9. A UV lamp assembly comprising:
a powerhead comprising:
a power cable;
a microswitch operatively connected to the power cable; and
a UV bulb socket; and
a mounting base that is releasably connectable to the powerhead, the mounting base comprising a ramp that activates the microswitch when the powerhead is connected to the mounting base.

10. The UV lamp assembly of claim 9, wherein the ramp comprises an inclined surface, and wherein the microswitch is configured to engage the ramp at multiple points along the inclined surface.

11. The UV lamp assembly of claim 9, wherein the microswitch comprises a lever with a distal end configured to traverse along the ramp.

12. The UV lamp assembly of claim 11, wherein the distal end comprises a roller.

13. A UV lamp pre-assembly comprising:
an HVAC system comprising a plenum at least partially enclosed by a wall, wherein the wall comprises a first through-hole;
a mounting base that is coupled to the wall and that comprises:
a plate comprising a back face, which is oriented towards the wall, and a front face that, relative to the back face, is on an opposite side of the plate; and
a second through-hole extending from the front face to the back face and at least partially aligned with the first through-hole; and
a cap that is attached to the mounting base and that covers the second through-hole.

14. The UV lamp pre-assembly of claim 13, further comprising a gasket that has a front face, which is oriented towards the plate, and a back face, relative to the front face, is on an opposite side of the plate.

15. The UV lamp pre-assembly of claim 14, wherein the gasket comprises a third through-hole.

16. The UV lamp pre-assembly of claim 15, wherein the first through-hole, the second through-hole, and the third through-hole are aligned.

17. The UV lamp pre-assembly of claim 14, further comprising:
the back face of the plate positioned against the front face of the gasket;
the back face of the gasket positioned against the wall; and
at least one fastener connecting the plate, the gasket, and the wall.

18. The UV lamp pre-assembly of claim 13, wherein a wall that extends from the second through-hole and includes an internal side towards the second through-hole and an external side away from the second through-hole.

19. The UV lamp pre-assembly of claim 18, wherein the external side of the wall comprises one or more mounting flanges that are externally threaded.

20. The UV lamp pre-assembly of claim 19, wherein the cap is attachable to the one or more mounting flanges.

21. The UV lamp pre-assembly of claim 13, wherein the cap comprises a plug.

22. The UV lamp pre-assembly of claim 21, wherein the plug comprises at least two undercut ribs and is insertable into the second through-hole, wherein the at least two undercut ribs are attachable to a bottom of the second through-hole.
